(19) United States
(12) Patent Application Publication
(10) Pub. No.: US 2002/0169208 A1
(43) Pub. Date: Nov. 14, 2002

(54) Novel anticholinergic compounds and methods of use

(76) Inventor: Pascal Druzgala, Santa Rosa, CA (US)

Correspondence Address: David R. Saliwanchik, Saliwanchik, Lloyd & Saliwanchik, A Professional Association, 2421 N.W. 41st Street, Suite A-1, Gainesville, FL, 32606-6669, US

(21) Appl. No.: 10/116,202

(22) Filed: Apr. 3, 2002

Related U.S. Application Data
(60) Provisional application No. 60/281,134, filed on Apr. 3, 2001.
(60) Provisional application No. 60/350,516, filed on Jan. 18, 2002.

(51) Int. Cl. A61K 31/225; C07C 69/76

(52) U.S. Cl. 514/547; 560/76; 560/190

(57) ABSTRACT

In a preferred embodiment, the subject invention concerns novel analogs of oxybutynin. The present invention also concerns methods for synthesizing the oxybutynin analogs of the present invention. The invention also pertains to methods for treating patients suffering from incontinence and other conditions.

### CROSS-REFERENCE TO RELATED APPLICATION

This application claims the benefit of U.S. Provisional Application Ser. Nos. 60/281,134, filed Apr. 3, 2001 and 60/350,516; filed Jan. 18, 2002.

### BACKGROUND OF INVENTION

Anticholinergic (cholinergic blocking) agents prevent, or diminish the ability of, the neurotransmitter acetylcholine from combining with receptors on the postganglionic parasympathetic nerve terminal (muscarinic site). Acetycholine, in effect, counteracts the effect of dopamine in the brain.

Effects of anticholinergic agents include reduction of smooth muscle spasms, blockade of vagal impulses to the heart, decreased secretions (e.g., gastric, salivation, bronchial mucus, seat glands), production of mydriasis and cycloplegia, and various central nervous system (CNS) effects. In therapeutic doses, these drugs have little effect on transmission of never impulses across ganglia (nicotinic sites) or at the neuromuscular junction. Several anticholinergic drugs abolish or reduce the symptoms of Parkinson's disease, such as tremors and rigidity, and result in improvement in mobility, muscular coordination, and motor performance. These effects may be due to blockade of the effects of acetylcholine in the CNS.

Commercially available anticholinergic drugs share a variety of undesirable side effects. These side effects can include: dry mouth, dysphagia, constipation, heartburn, change in taste perception, bloated feeling, paralytic ileus, dizziness, drowsiness, nervousness, disorientation, headache, weakness, insomnia, urinary retention or hesitancy, impotence, blurred vision, dilated pupils, photophobia, cycloplegia, precipitation acute glaucoma, flushing, decreased sweating, nasal congestion, and suppression of glandular secretions including lactation. Large doses may produce CNS stimulation including tremor and restlessness.

In view of the side effects of existing anticholinergic compounds a variety of efforts have been made to create analogs of existing compounds and/or to identify new anticholinergic compounds. See, for example, U.S. Pat. No. 5,637,601 and references cited therein.

Anticholinergic agents have been used, or show potential for use, in a variety of therapeutic applications including, use as antiperspirants, mydriatic agents and for the treatment of a variety of ailments including respiratory conditions, including obstructive pulmonary diseases; incontinence; and Parkinson's disease.

Mydriatic agents are an important class of compounds that are used to dilate the pupil. Mydriasis is required during ophthalmic examinations, in order to provide for a more complete examination of the fundus, the vitreous and the periphery of the lens, and in various surgical procedures. Commercially available mydriatic drugs such as atropine, scopolamine, and homatropine suffer from several disadvantages. Because the mydriasis induced by these agents causes blurred vision and is of a relatively long duration, i.e., several hours, it is necessary to virtually immobilize the patient after the ophthalmic examination until the mydriasis subsides and the patient can resume normal activities. Ophthalmic use of these agents may also induce local side effects such as transient stinging, allergic lid reactions, follicular conjunctivitis, edema and photophobia.

With regard to the use of anticholinergic compounds as antiperspirants, systemically administered anticholinergics do generally decrease the secretion of the sweat glands as well as saliva and that of other secretory glands. Based on these properties, it has been investigated how antimuscarinic agents could be used to inhibit local hyperhydration by topical application. A major problem in employing anticholinergic compounds heretofore for the control of human perspiration revolves about the mydriatic activity of such compounds on the eye. While it is possible to reduce the risk of mydriasis by reducing to a minimum the concentration of the active anticholinergic ingredient, it has generally been felt that the risks have outweighed the benefits.

Asthma, bronchitis and emphysema are known as Chronic Obstructive Pulmonary Diseases (COPD). COPD is characterized as generalized airways obstruction, particularly of small airways, associated with varying degrees of symptoms of chronic bronchitis, asthma, and emphysema. The term COPD was introduced because these conditions often coexist, and it may be difficult in an individual case to decide which is the major condition producing the obstruction. Airways obstruction is defined as an increased resistance to airflow during forced expiration. It may result from narrowing of airways secondary to intrinsic airways disease, from excessive collapse of airways during a forced expiration secondary to pulmonary emphysema, from bronchospasm as in asthma, or may be due to a combination of these factors.

Asthma is characterized by increased responsiveness of the airway, resulting in airway obstruction. The underlying mechanisms causing asthma are unknown, but inherited or acquired imbalance of adrenergic and cholinergic control of airway diameter has been implicated. Overt asthma attacks may occur when individuals are subjected to various stresses, such as viral respiratory infection, exercise, emotional upset, nonspecific factors (e.g., changes in barometric pressure or temperature), inhalation of cold air or irritants (e.g., gasoline fumes, fresh paint and noxious odors, or cigarette smoke), exposure to specific allergens, and ingestion of aspirin or sulfites in sensitive individuals. In many persons, both allergenic and non-allergenic factors are significant.

Anticholinergic drugs block the action of the neurotransmitter acetylcholine on neurons in the brain. Normally, acetylcholine and dopamine have opposite effects, at least in the motor areas of the brain. Because the level of dopamine is reduced in Parkinson's patients, the neurons responsible for smooth motor control become overstimulated by acetylcholine, causing tremors and rigidity. However, anticholinergic drugs decrease the influence of acetylcholine in the body, either by preventing its production, blocking its receptor sites, or breaking it down chemically. This helps to restore the chemical balance between dopamine and acetylcholine in the motor system.

Many people are affected by urinary incontinence. Incontinence is particularly common in the elderly, urinary incontinence is present in approximately fifty percent of nursing home patients, and urinary incontinence is a well known urologic problem in women. It will affect nearly all women in some form during their lifetime, and it is of significant medical and social concern to all humans who experience it.

Involuntary incontinence also known as urge incontinence and overactive bladder, occurs with a loss of a large volume of urine accompanied by symptoms of urgency, frequency and nocturia caused by an unstable bladder or detrusor instability. The patient may lose urine with a change in position or with auditory stimulation. The loss of small volumes of urine usually occurs because bladder over distension by a large amount of residual urine referred to as overflow incontinence.

The present management of incontinence consists in administering a muscle relaxant, such as oxybutynin, which acts directly on the smooth muscle at the site distal to the cholinergic receptor. The usual dose for the pharmacologic management of incontinence is repeated, nonsustained and noncontrolled doses from two-to-four times a day for oxybutynin. Steriods, estrogen and/or progesterone hormone replacement therapy have also been used, however, this therapy is typically insufficient for the management of incontinence.

Oxybutynin (Ditropan®) is a relatively non-specific anticholinergic agent that is used in the treatment of incontinence and intestinal hypermotility. Chemical names for oxybutynin are 4-(diethylamino)-2-butynyl-.alpha.-cyclohexyl-.alpha.-hydroxy benzeneacetate, and 4-(diethylamino)-2-butynylphenylcyclohexyl-glycolate. It is a racemic mixture of the R-enantiomer, R-oxybutynin, and the S-enantiomer, S-oxybutynin. Use of the S-enantiomer of oxybutynin, S-oxybutynin, for the treatment of urinary incontinence has been described in U.S. Pat. Nos. 5,532,278, and 5,736,577.

Administration of racemic oxybutynin may result in a number of adverse effects. These adverse effects include, but are not limited to, xerostomia, mydriasis, drowsiness, nausea, constipation, palpitations and tachycardia. The amelioration of cardiovascular side effects of racemic oxybutynin, such as tachycardia and palpitations, is of particular therapeutic value.

Oxybutynin is a widely used drug despite a marginal activity and inconvenient side effects. One of the most common side effects encountered with oxybutynin is the inhibitory action on the salivery glands, which is responsible for the “dry mouth” symptom. This is possibly due to its affinity for the M₅ muscarinic receptor. The primary metabolite, the N-desethylated analog, has even higher affinity for this receptor subtype. An oxybutynin analog having rapid deactivation rate by non-oxidative pathways to an inactive primary metabolite would therefore be highly desirable.

### BRIEF SUMMARY OF THE INVENTION

The subject invention provides anticholinergic agents which are useful in the treatment of a variety of conditions, including incontinence. In further embodiments, the compounds of the subject invention can be used as mydriatic agents and antiperspirants.

The present invention also provides methods for synthesizing oxybutynin analogs.

The present invention also provides methods of treatment which involve administering an effective amount of a compound of the present invention to a person in need of such treatment.

### BRIEF DESCRIPTION OF DRAWINGS

FIG. 1 shows the oxybutynin molecule has at least two potential sites (indicated by arrows) where transformation techniques can be applied.

FIG. 2 shows a first approach to creating novel anticholinergic molecules according to the present invention where the inactive metabolite is a monoester of 2-cyclohehyl-2-phenylmalonic acid.

FIG. 3 shows the formation of a reverse ester analog to the oxybutynin structure, resulting in two inactive metabolites upon hydrolytic cleavage by non-oxidative enzymes.

FIG. 4 shows the formation of a carbonate analog to the oxybutynin structure.

FIGS. 5-7 show synthetic schemes for preparing compounds of the subject invention.

### DETAILED DISCLOSURE

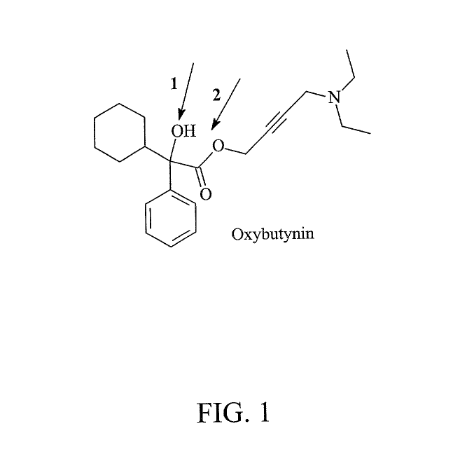

The subject invention provides new and advantageous compounds that have anti-cholinergic activity. In a specific embodiment of the subject invention, the compounds of the formula of General Structure I or as shown in FIG. 1 are useful for treating patients suffering from incontinence. Compounds of the subject invention can also be used for creating bronchodilation in patients suffering from asthma or obstructive airway disease. They can be used as mydriatic agents. In yet another embodiment, the compounds of the subject invention can be used as antiperspirants.

In a preferred embodiment, the subject invention provides novel analogs of oxybutynin that have less side effects than the parent molecule when administered to a patient. Advantageously, the compounds of the subject invention are rapidly deactivated by nonoxidative pathways. Preferably, the compounds have a lower affinity for the M₅ muscarinic receptor. In one embodiment, the oxybutynin analog of the subject invention is a reverse ester. In another embodiment, the oxybutynin analog of the subject invention is a carbonate analog of the oxybutynin structure. Optionally, in any of the compounds of the invention, the terminal nitrogen atom of the oxybutynin analog can be quaternized.

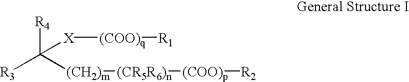

Compounds of the subject invention include those wherein:

R₁ is alkyl, alkenyl, alkynyl, cycloalkyl, cycloalkenyl, cycloalkynyl, or a combination thereof, containing at least one tertiary or quaternary nitrogen atom;

R₂ is H, OH, or C₁₋₄ alkyl;

R₃ and R4 are, independently, H, hydroxyl, alkyl, cycloalkyl, aryl, or heteroaryl, optionally substituted with lower alkyl, hydroxyl, hydroxymethyl, COOH, or COO-lower alkyl;

R₅ and R6 are independently H, C₁₋₄ alkyl, or R₅ and R₆ together form a cycloalkyl ring optionally containing a nitrogen or an oxygen atom, and optionally substituted with hydroxyl, hydroxymethyl, or lower alkyl;

m is an integer from 0 to 14;

n, p, and q are, independently, 0 or 1;

when m, n, and p=0, then R₂ and R₃ together can form a cycloalkyl ring optionally substituted with lower alkyl, COOH, or COO-lower alkyl; and

X is O, NH, S, CH₂, or X is a bond.

Adverse drug-drug interactions (DDI), elevation of liver function test (LFT) values, and QT prolongation leading to torsades de pointes (TDP) are three major reasons why drug candidates fail to obtain FDA approval. All these causes are, to some extent metabolism-based. A drug that has two metabolic pathways, one oxidative and one non-oxidative, built into its structure is highly desirable in the pharmaceutical industry. An alternate, non-oxidative metabolic pathway provides the treated subject with an alternative drug detoxification pathway (an escape route) when one of the oxidative metabolic pathways becomes saturated or non-functional. While a dual metabolic pathway is necessary in order to provide an escape metabolic route, other features are needed to obtain drugs that are safe regarding DDI, TDP, and LFT elevations.

In addition to having two metabolic pathways, the drug should have a rapid metabolic clearance (short metabolic half-life) so that blood levels of unbound drug do not rise to dangerous levels in cases of DDI at the protein level. Also, if the metabolic half-life of the drug is too long, then the CYP450 system again becomes the main elimination pathway, thus defeating the original purpose of the design. In order to avoid high peak concentrations and rapidly declining blood levels when administered, such a drug should also be administered using a delivery system that produces constant and controllable blood levels over time.

The compounds of this invention have one or more of the following characteristics or properties:

1. Compounds of the invention are metabolized both by CYP450 and by a non-oxidative metabolic enzyme or system of enzymes;

2. Compounds of the invention have a short (up to four (4) hours) non-oxidative metabolic half-life;

3. Oral bioavailability of the compounds is consistent with oral administration using standard pharmaceutical oral formulations; however, the compounds, and compositions thereof, can also be administered using any delivery system that produces constant and controllable blood levels over time;

4. Compounds according to the invention contain a hydrolysable bond that can be cleaved non-oxidatively by hydrolytic enzymes;

5. Compounds of the invention can be made using standard techniques of small-scale and large-scale chemical synthesis;

6. The primary metabolites of compounds of this invention results from the non-oxidative metabolism of the compounds;

7. The primary metabolites, regardless of the solubility properties of the parent drug, is, or are, soluble in water at physiological pH and have, as compared to the parent compound, a significantly reduced pharmacological activity;

8. The primary metabolites, regardless of the electrophysiological properties of the parent drug, has, or have, negligible inhibitory activity at the IK_{R} (HERG) channel at normal therapeutic concentration of the parent drug in plasma (e.g., the concentration of the metabolite must be at least five times higher than the normal therapeutic concentration of the parent compound before activity at the IK_{R} channel is observed);

9. Compounds of the invention, as well as the metabolites thereof, do not cause metabolic DDI when co-administered with other drugs;

10. Compounds of the invention, as well as metabolites thereof, do not elevate LFT values when administered alone.

In some embodiments, the subject invention provides compounds have any two of the above-identified characteristics or properties. Other embodiments provide for compounds having at least any three of the above-identified properties or characteristics. In another embodiment, the compounds, and compositions thereof, have any combination of at least four of the above-identified characteristics or properties. Another embodiment provides compounds have any combination of five to 10 of the above-identified characteristics or properties. In a preferred embodiment the compounds of the invention have all ten characteristics or properties.

In various embodiments, the primary metabolites of the inventive compounds, regardless of the electrophysiological properties of the parent drug, has, or have, negligible inhibitory activity at the IK_{R} (HERG) channel at normal therapeutic concentrations of the drug in plasma. In other words, the concentration of the metabolite must be at least five times higher than the normal therapeutic concentration of the parent compound before activity at the IK_{R} channel is observed. Preferably, the concentration of the metabolite must be at least ten times higher than the normal therapeutic concentration of the parent compound before activity at the IK_{R} channel is observed.

Compounds according to the invention are, primarily, metabolized by endogenous hydrolytic enzymes via hydrolysable bonds engineered into their structures. The primary metabolites resulting from this metabolic pathway are water soluble and do not have, or show a reduced incidence of, DDI when administered with other medications (drugs). Non-limiting examples of hydrolysable bonds that can be incorporated into compounds according to the invention include amide, ester, carbonate, phosphate, sulfate, urea, urethane, glycoside, or other bonds that can be cleaved by hydrolases.

Additional modifications of the compounds disclosed herein can readily be made by those skilled in the art. Thus, analogs and salts of the exemplified compounds are within the scope of the subject invention. With a knowledge of the compounds of the subject invention skilled chemists can use known procedures to synthesize these compounds from available substrates. As used in this application, the term “analogs” refers to compounds which are substantially the same as another compound but which may have been modified by, for example, adding additional side groups. The term “analogs” as used in this application also may refer to compounds which are substantially the same as another compound but which have atomic or molecular substitutions at certain locations in the compound.

The present invention also concerns methods for synthesizing the oxybutynin analogs of the present invention. The oxybutynin chemical structure lends itself to several types of transformations. Two transformations exemplified herein are those leading to reverse esters and to carbonate analogs. In addition to these transformations, the terminal nitrogen atom can be quaternized in order to avoid possible central side effects on the brain muscarinic receptors. Quaternization of the nitrogen is, however, not necessary in order to achieve the desired activity and characteristics of the compounds of the subject invention. Using these kinds of transformations, analogs produced thereby can be rapidly screened by muscarinic receptor binding in vitro.

As shown in FIG. 1, the oxybutynin molecule has at least two potential sites (indicated by arrows) where transformation techniques can be applied. In addition to these sites, a positive charge can be introduced on the nitrogen in order to keep the molecule from crossing the blood-brain barrier. This results in the loss of side effects that are due to central effects. This also results in a lower affinity for the M₅ receptor.

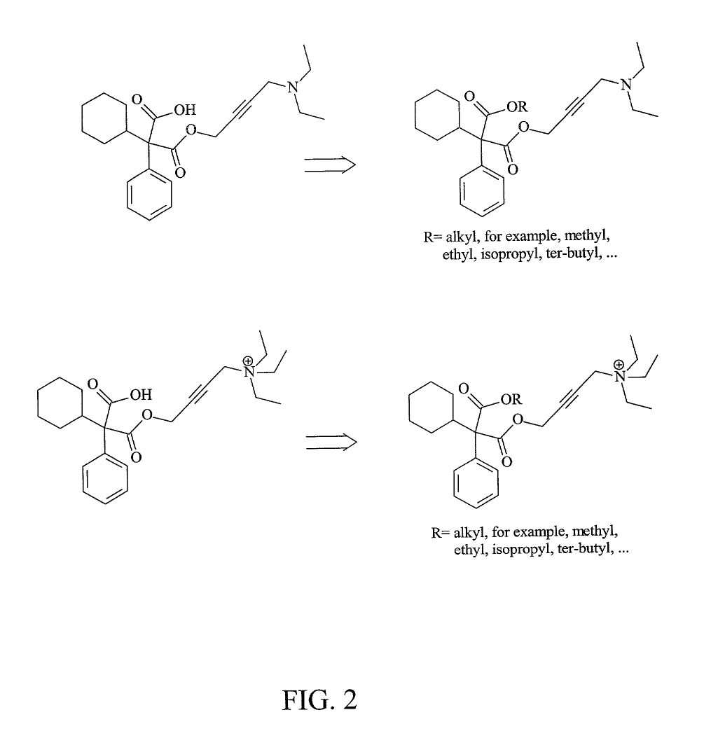

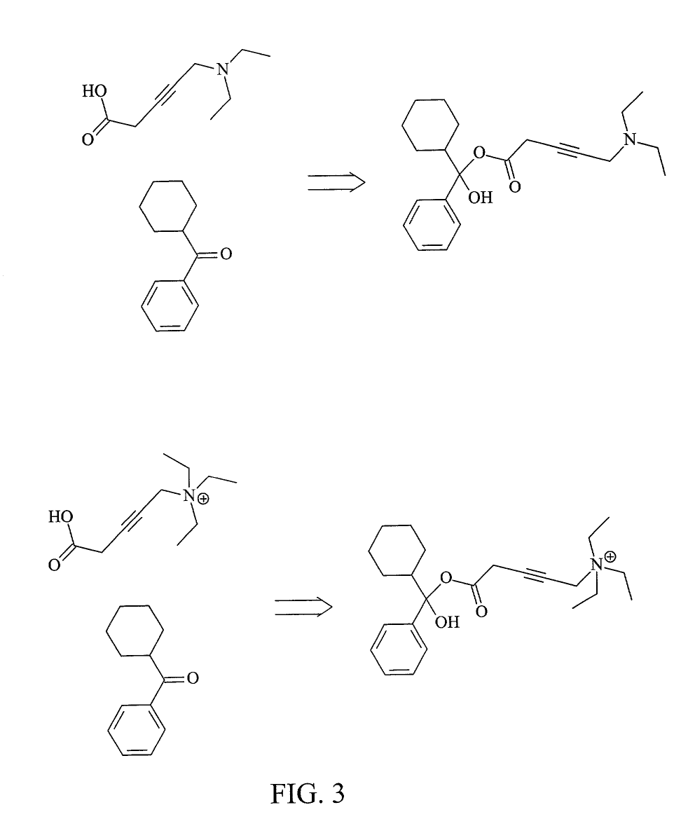

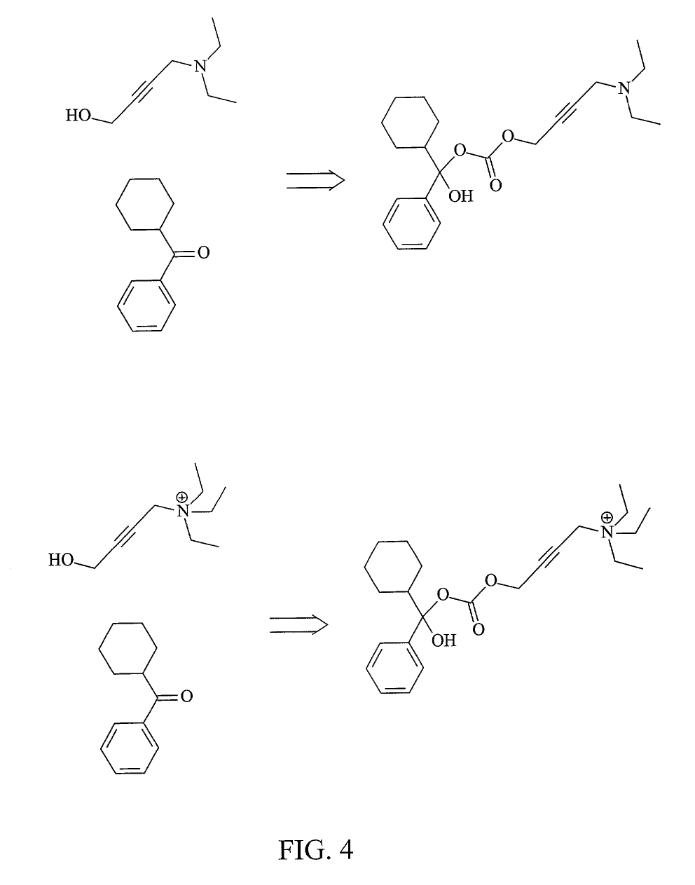

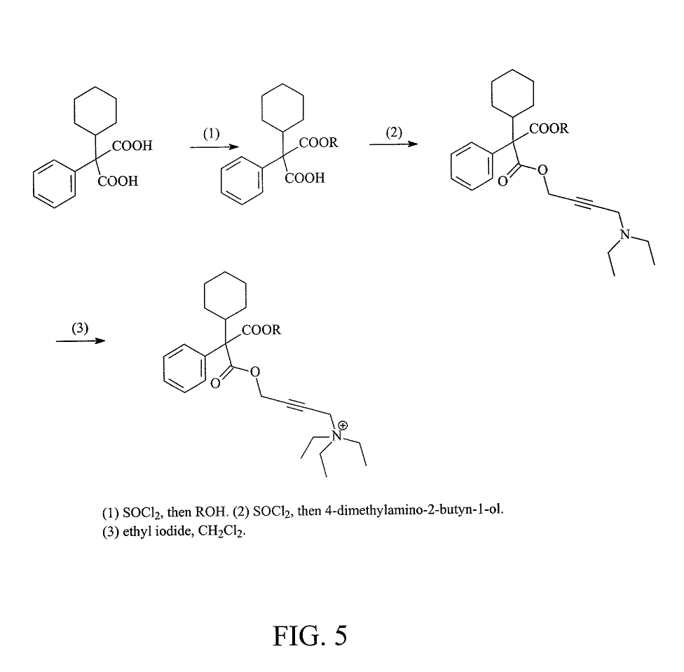

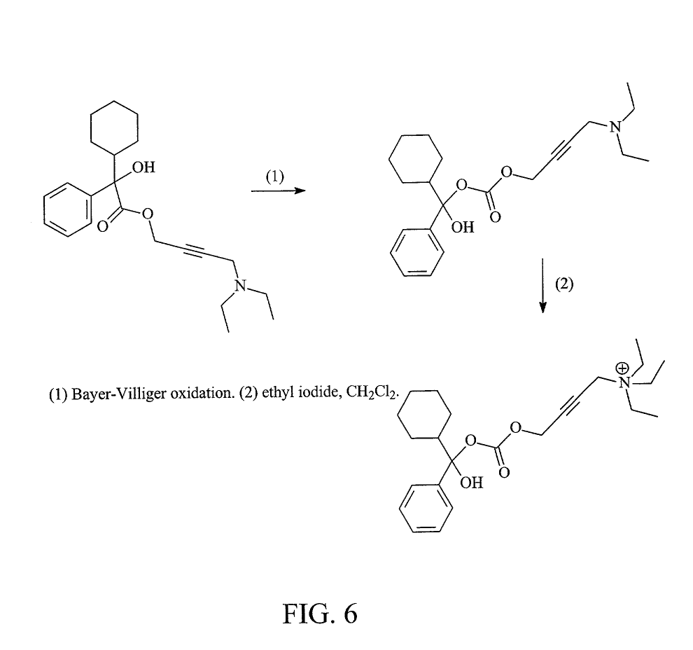

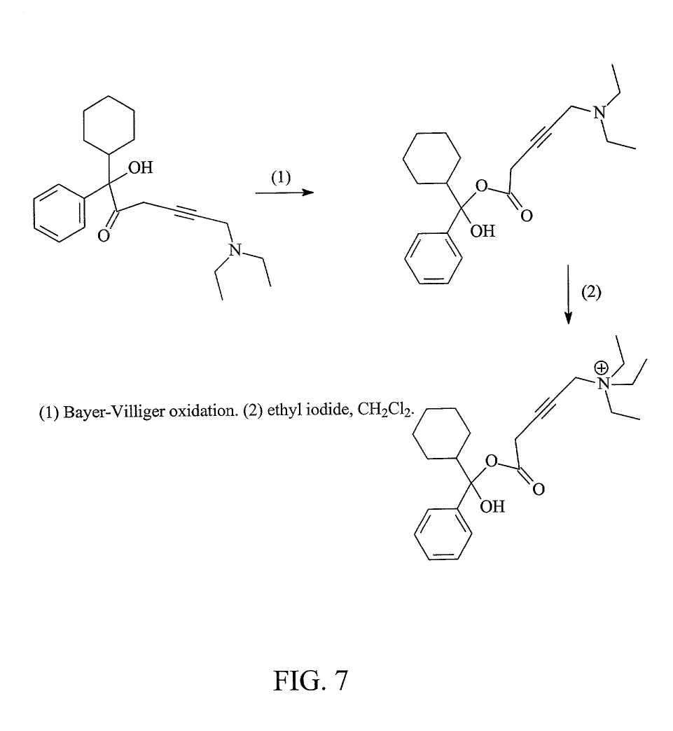

A first approach to creating novel anticholinergic molecules according to the present invention is shown in FIG. 2, where the inactive metabolite is a monoester of 2-cyclohehyl-2-phenylmalonic acid. Another approach is through the formation of a reverse ester analog to the oxybutynin structure (FIG. 3), resulting in two inactive metabolites upon hydrolytic cleavage by non-oxidative enzymes. A third approach (FIG. 4) is through the formation of a carbonate analog to the oxybutynin structure. Synthetic schemes for preparing these molecules are shown in FIGS. 5, 6, and 7.

The invention also concerns methods of using the present compounds to treat incontinence in a patient. The compounds can be delivered by various methods and routes known in the art. Preferably, the compounds are delivered via transdermal or transmucosal means.

The compounds of this invention have therapeutic properties similar to those of the unmodified parent compounds. Accordingly, dosage rates and routes of administration of the compounds of the subject invention are similar to those already used in the art and known to the skilled artisan (see, for example, Physicians' Desk Reference, 54^{th} Ed., Medical Economics Company, Montvale, N.J., 2000).

The compounds of the subject invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention are formulated such that an effective amount of the bioactive compound(s) is combined with a suitable carrier in order to facilitate effective administration of the composition.

In accordance with the subject invention, pharmaceutical compositions are provided which comprise, as an active ingredient, an effective amount of one or more of the compounds and one or more non-toxic, pharmaceutically acceptable carriers or diluents. Examples of such carriers for use in the subject invention include ethanol, dimethyl sulfoxide, glycerol, silica, alumina, starch, and equivalent carriers and diluents.

Further, acceptable carriers can be either solid or liquid. Solid form preparations include powders, tablets, pills, capsules, cachets, suppositories and dispersible granules. A solid carrier can be one or more substances which may act as diluents, flavoring agents, solubilizers, lubricants, suspending agents, binders, preservatives, tablet disintegrating agents or encapsulating materials.

The disclosed pharmaceutical compositions may be subdivided into unit doses containing appropriate quantities of the active component. The unit dosage form can be a packaged preparation, such as packeted tablets, capsules, and powders in paper or plastic containers or in vials or ampoules. Also, the unit dosage can be a liquid based preparation or formulated to be incorporated into solid food products, chewing gum, or lozenge.

The compounds of the subject invention can be used to treat human and other animals.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. A more complete understanding of the invention can be obtained by reference to the following specific examples of compounds of the invention. It will be apparent to those skilled in the art that the examples involve use of materials and reagents that are commercially available from known sources, e.g., chemical supply houses, so no details are given respecting them. These examples should not be construed as limiting. All percentages are by weight and all solvent mixture proportions are by volume unless otherwise noted.

The following compounds exemplify the anticholinergic compounds of the subject invention:

### EXAMPLE 1

### m, n, p, and q=0, X is a bond, and R₂ is hydrogen

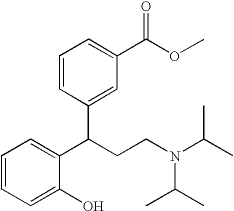

### Structure II 3-[3-Diisopropylamino-1-(2-hydroxy-phenyl)-propyl]-benzoic acid methyl ester

Other examples include:

3-[1-(2-Hydroxy-phenyl)-3-pyrrolidin-1-yl-propyl]-benzoic acid methyl ester;

3-(1-Cyclohexyl-3-diisopropylaminopropyl)-4-hydroxy-benzoic acid methyl ester;

Carboxymethyl-[3-cyclohexyl-3-(2-hydroxy-5-methyl-phenyl)-propyl]-diisopropyl-ammonium;

Methoxycarbonylmethyl-[3-cyclohexyl-3-(2-hydroxy-5-methyl-phenyl)-propyl]-diisopropyl-ammonium;

Methoxycarbonylmethyl-[3-(2-hydroxy-5-methyl-phenyl)-3-phenyl-propyl]-diisopropyl-ammonium; and

Methoxycarbonylmethyl-[3-(5-acetoxymethyl-2-hydroxy-phenyl)-3-phenyl-propyl]-diisopropyl-ammonium.

### EXAMPLE 2

### m, n p, and q=0, X is a bond, and R₂ is hydroxyl

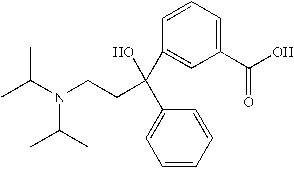

### Structure III. 3-(3-Diisopropylamino-1-hydroxy-1-phenyl-propyl)-benzoic acid

Other examples include:

3-(3-Diisopropylamino-1-hydroxy-1-phenyl-propyl)-benzoic acid methyl ester;

3 -Diisopropylamino-1-(3-hydroxymethyl-phenyl)-1-phenyl-propan-1-ol;

3 -Diisopropylamino-1-phenyl-1-m-tolyl-propan-1-ol;

Carboxymethyl-(3-hydroxy-3-phenyl-3-m-tolyl-propyl)-diisopropyl-ammonium; and (3-Hydroxy-3-phenyl-3-m-tolyl-propyl)-diisopropyl-methoxycarbonylmethyl-ammonium.

### EXAMPLE 3

### m, n, p, and q=0, X is CH₂, and R₂ is hydroxyl

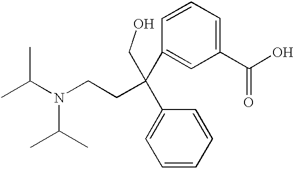

### Structure IV. 3-(3-Diisopropylamino-1-hydroxymethyl-1-phenyl-propyl)-benzoic acid

Other examples include:

3-(3-Diisopropylamino-1-hydroxymethyl-1-phenyl-propyl)-benzoic acid methyl ester;

4-Diisopropylamino-2-(3-hydroxymethyl-phenyl)-2-phenyl-butan-1-ol;

4-Diisopropylamino-2-phenyl-2-m-tolyl-butan-1-ol;

Carboxymethyl-(3-hydroxymethyl-3-phenyl-3-m-tolyl-propyl)-diisopropyl-ammonium; and

Methoxycarbonylmethyl-(3-hydroxymethyl-3-phenyl-3-m-tolyl-propyl)-diisopropyl-ammonium.

### EXAMPLE 4

### m, n, and q=0, p=1, X is a bond, and R₂ is hydrogen or lower alkyl

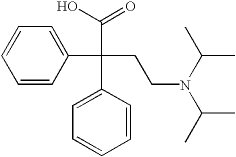

### Figure V. 4-Diisopropylamino-2,2-diphenyl-butyric acid

Other examples include:

4-Diisopropylamino-2,2-diphenyl-butyric acid methyl ester'

4-Diisopropylamino-2,2-diphenyl-butyric acid ethyl ester;

4-Diisopropylamino-2-phenyl-2-m-tolyl-butyric acid ethyl ester;

4-Diisopropylamino-2-(3-hydroxymethyl-phenyl)-2-phenyl-butyric acid ethyl ester;

4-Diisopropylamino-2-(3-hydroxymethyl-phenyl)-2-phenyl-butyric acid isopropyl ester; and

2-(3-Acetoxymethyl-phenyl)-4-diisopropylamino-2-phenyl-butyric acid methyl ester

### EXAMPLE 5

### m=1,n=0 or 1, p=1, q=0, X is a bond and R₂ is hydrogen or lower alkyl

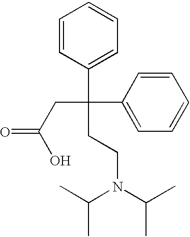

### Structure VI. 5-Diisopropylamino-3,3-diphenyl-pentanoic acid

Other examples include:

5-Diisopropylamino-3,3-diphenyl-pentanoic acid methyl ester;

5-Diisopropylamino-3,3-diphenyl-pentanoic acid ethyl ester;

5-Diethylamino-3,3-diphenyl-pentanoic acid ethyl ester;

5-Diethylamino-2-methyl-3,3-diphenyl-pentanoic acid ethyl ester;

5-Diethylamino-2,2-dimethyl-3,3-diphenyl-pentanoic acid ethyl ester;

3-Cyclopentyl-5-diethylamino-2,2-dimethyl-3-phenyl-pentanoic acid ethyl ester;

3-Cyclohexyl-5-diethylamino-2,2-dimethyl-3-phenyl-pentanoic acid ethyl ester;

5-Diethylamino-3-hydroxy-2,2-dimethyl-3-phenyl-pentanoic acid ethyl ester;

1-(3-Diethylamino-1-hydroxy-1-phenyl-propyl)-cyclopentanecarboxylic acid methyl ester;

1-(3-Diethylamino-1-hydroxy-1-phenyl-propyl)-cyclohexanecarboxylic acid methyl ester;

1-(3-Diethylamino-1-hydroxy-1-phenyl-propyl)-cyclopropanecarboxylic acid methyl ester;

1-(5-Diethylamino-1-hydroxy-1-phenyl-pent-3-ynyl)-cyclohexanecarboxylic acid methyl ester;

1-(1-Hydroxy-1-phenyl-5-pyrrolidin-1-yl-pent-3-ynyl)-cyclohexanecarboxylic acid methyl ester; and

1-(1-Hydroxy-1-phenyl-5-pyrrolidin-1-yl-pent-3-ynyl)-cyclopentanecarboxylic acid methyl ester.

### EXAMPLE 6

### m, n, and p=0 R₂ is hydroxyl, X is a bond, and q=1

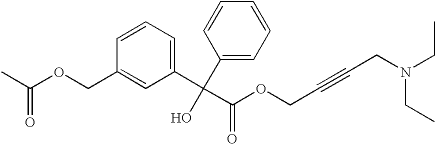

### Structure VII. (3-Acetoxymethyl-phenyl)-hydroxy-phenyl-acetic acid 4-diethylamino-but-2-ynyl ester

Other examples include:

3-[(4-Diethylamino-but-2-ynyloxycarbonyl)-hydroxy-phenyl-methyl]-benzoic acid;

3-[(4-Diethylamino-but-2-ynyloxycarbonyl)-hydroxy-phenyl-methyl]-benzoic acid methyl ester;

3-[Hydroxy-(8-methyl-8-aza-bicyclo[3.2.1]oct-3-yloxycarbonyl)-phenyl-methyl]-benzoic acid methyl ester;

3-[2-Hydroxy-2-(3-methoxycarbonyl-phenyl)-2-phenyl-acetoxy]-8,8-dimethyl-8-azonia-bicyclo[3.2.1]octane; and

3-[(1-Aza-bicyclo[2.2.2]oct-3-yloxycarbonyl)-hydroxy-phenyl-methyl]-benzoic acid methyl ester;

### EXAMPLE 7

### m n, and p=0 and R₂ and R₃ together form a cycloalkyl group

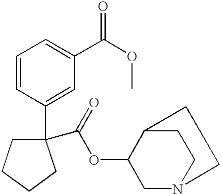

### Structure VIII. 3-[1-(1-Aza-bicyclo[2.2.2]oct-3-yloxycarbonyl)-cyclopentyl]-benzoic acid methyl ester

Other examples include:

3-[1-(1-Aza-bicyclo[2.2.2]oct-3-yloxycarbonyl)-cyclopentyl]-4-hydroxy-benzoic acid methyl ester;

3-[1-(1-Aza-bicyclo[2.2.2]oct-3-yloxycarbonyl)-cyclohexyl]-4-hydroxy-benzoic acid methyl ester;

3-[1-(4-Diethylamino-but-2-ynyloxycarbonyl)-cyclohexyl]-4-hydroxy-benzoic acid methyl ester;

3-[1-(4-Diethylamino-but-2-ynyloxycarbonyl)-cyclopentyl]-4-hydroxy-benzoic acid methyl ester;

4-Hydroxy-3-[1-(8-methyl-8-aza-bicyclo[3.2.1]oct-3-yloxycarbonyl)-cyclopentyl]-benzoic acid methyl ester; and

3-[1-(2-Hydroxy-5-methoxycarbonyl-phenyl)-cyclopentanecarbonyloxy]-8,8-dimethyl-8-azonia-bicyclo[3.2.1]octane.

### EXAMPLE 8

### X is not a bond

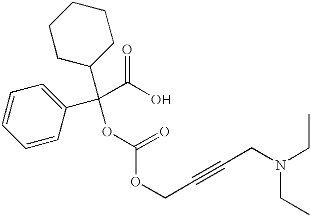

### Structure IX. Cyclohexyl-(4-diethylamino-but-2-ynyloxycarbonyloxy)-phenyl-acetic acid

Other examples include:

Cyclohexyl-(4-diethylamino-but-2-ynyloxycarbonyloxy)-phenyl-acetic acid methyl ester;

Cyclohexyl-(4-diethylamino-but-2-ynyloxycarbonylamino)-phenyl-acetic acid methyl ester;

Cyclohexyl-(4-diethylamino-but-2-ynyloxycarbonylsulfan yl)-phenyl-acetic acid methyl ester;

(4-Diethylamino-but-2-ynyloxycarbonyloxy)-phenyl-acetic acid methyl ester;

(4-Diethylamino-but-2-ynyloxycarbonyloxy)-phenyl-acetic acid ethyl ester;

Phenyl-(4- pyrrolidin-1-yl-but-2-ynyloxycarbonyloxy)-acetic acid ethyl ester;

(8-Methyl-8-aza-bicyclo[3.2.1]oct-3-yloxycarbonyloxy)-phenyl-acetic acid ethyl ester;

3-(Ethoxycarbonyl-phenyl-methoxycarbonyloxy)-8,8-dimethyl-8-azonia-bicyclo[3.2.1]octane;

[(1-Aza-bicyclo[2.2.2]oct-3-yloxycarbonyloxy)]-phenyl-acetic acid ethyl ester;

3-(Ethoxycarbonyl-phenyl-methoxycarbonyloxy)-1-methyl-1-azonia-bicyclo[2.2.2]octane;

3-[2-Hydroxy-1-(3-methoxycarbonyl-phenyl)-ethoxycarbonyloxy]-1-methyl-1-azonia-bicyclo[2.2.2]octane;

3-[1-(1-Aza-bicyclo[2.2.2]oct-3-yloxycarbonyloxy)-2-hydroxy-ethyl]-benzoic acid methyl ester;

3-[2-Hydroxy-1-(8-methyl-8-aza-bicyclo[3.2.1]oct-3-yloxycarbonyloxy)-ethyl]-benzoic acid methyl ester; and

3-[2-Hydroxy-1-(3-methoxycarbonyl-phenyl)-ethoxycarbonyloxy]-8,8-dimethyl-8-azonia-bicyclo[3.2.1]octane.

It should be understood that the examples and embodiments described herein are for illustrative purposes only and that various modifications or changes in light thereof will be suggested to persons skilled in the art and are to be included within the spirit and purview of this application.

I claim:

1. An anticholinergic compound having at least one of the following characteristics:
a. the compound is metabolized both by CYP450 and by a non-oxidative metabolic enzyme or system of enzymes;
b. the compound has a short (up to four (4) hours) non-oxidative metabolic half-life;
c. the compound contains a hydrolysable bond that can be cleaved non-oxidatively by hydrolytic enzymes;
d. the primary metabolites of the compound result from the non-oxidative metabolism of the compound;
e. the primary metabolites are soluble in water at physiological pH;
f. the primary metabolites have negligible inhibitory activity at the IK_{R} (HERG) channel at normal therapeutic concentration of the parent drug in plasma;
g. the compound, as well as the metabolites thereof, does not cause metabolic DDI when co-administered with other drugs; and
h. the compound, as well as metabolites thereof, does not elevate LFT values when administered alone.

2. The anticholinergic compound, according to claim 1, having the following formula: 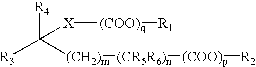
wherein:
R₁ is alkyl, alkenyl, alkynyl, cycloalkyl, cycloalkenyl, cycloalkynyl, or a combination thereof, containing at least one tertiary or quaternary nitrogen atom;
R₂ is H, OH, or C₁₋₄ alkyl;
R₃ and R₄ are, independently, H, hydroxyl, alkyl, cycloalkyl, aryl, or heteroaryl, optionally substituted with lower alkyl, hydroxyl, hydroxymethyl, COOH, or COO-lower alkyl;
R₅ and R₆ are independently H, C₁₋₄ alkyl, or R₅ and R₆ together form a cycloalkyl ring optionally containing a nitrogen or an oxygen atom, and optionally substituted with hydroxyl, hydroxymethyl, or lower alkyl;
m is an integer from 0 to 14;
n, p, and q are, independently, 0 or 1;
when m, n, and p=0, then R₂ and R₃ together can form a cycloalkyl ring optionally substituted with lower alkyl, COOH, or COO-lower alkyl; and
X is O, NH, S, CH₂, or X is a bond.

3. The compound, according to claim 2, wherein m, n, p, and q=0, X is a bond and R₂ is hydroxyl or hydrogen.

4. The compound, according to claim 3, wherein said compound is selected from the group consisting of 3-[3-Diisopropylamino-1-(2-hydroxy-phenyl)-propyl]-benzoic acid methyl ester;
3-[1-(2-Hydroxy-phenyl)-3-pyrrolidin-1-yl-propyl]-benzoic acid methyl ester;
3-(1-Cyclohexyl-3-diisopropylaminopropyl)-4-hydroxy-benzoic acid methyl ester;
Carboxymethyl-[3-cyclohexyl-3-(2-hydroxy-5-methyl-phenyl)-propyl]-diisopropyl-ammonium;
Methoxycarbonylmethyl-[3-cyclohexyl-3-(2-hydroxy-5-methyl-phenyl)-propyl]-diisopropyl-ammonium;
Methoxycarbonylmethyl-[3-(2-hydroxy-5-methyl-phenyl)-3-phenyl-propyl]-diisopropyl-ammonium;
Methoxycarbonylmethyl-[3-(5-acetoxymethyl-2-hydroxy-phenyl)-3-phenyl-propyl]-diisopropyl-ammonium.
3-(3-Diisopropylamino-1-hydroxy-1-phenyl-propyl)-benzoic acid;
3-(3-Diisopropylamino-1-hydroxy-1-phenyl-propyl)-benzoic acid methyl ester;
3-Diisopropylamino-1-(3-hydroxymethyl-phenyl)-1-phenyl-propan-1-ol;
3-Diisopropylamino-1-phenyl-1-m-tolyl-propan-1-ol;
Carboxymethyl-(3-hydroxy-3-phenyl-3-m-tolyl-propyl)-diisopropyl-ammonium; and
(3-Hydroxy-3-phenyl-3-m-tolyl-propyl)-diisopropyl-methoxycarbonylmethyl-ammonium.

5. The compound, according to claim 2, wherein m, n, p, and q=0; X is CH₂; and R₂ is hydroxyl.

6. The compound, according to claim 5, wherein said compound is selected from the groups consisting of 3-(3-Diisopropylamino-1-hydroxymethyl-1-phenyl-propyl)-benzoic acid;
3-(3-Diisopropylamino-1-hydroxymethyl-1-phenyl-propyl)-benzoic acid methyl ester;
4-Diisopropylamino-2-(3-hydroxymethyl-phenyl)-2-phenyl-butan-1-ol;
4-Diisopropylamino-2-phenyl-2-m-tolyl-butan-1-ol;
Carboxymethyl-(3-hydroxymethyl-3-phenyl-3-m-tolyl-propyl)-diisopropyl-ammonium; and
Methoxycarbonylmethyl-(3-hydroxymethyl-3-phenyl-3-m-tolyl-propyl)-diisopropyl-ammonium.

7. The compound, according to claim 2, wherein m, n, and q=0; p=1; X is a bond; and R₂ is hydrogen or lower alkyl.

8. The compound, according to claim 7, wherein said compound is selected from the group consisting of 4-Diisopropylamino-2,2-diphenyl-butyric acid;
4-Diisopropylamino-2,2-diphenyl-butyric acid methyl ester'
4-Diisopropylamino-2,2-diphenyl-butyric acid ethyl ester;
4-Diisopropylamino-2-phenyl-2-m-tolyl-butyric acid ethyl ester;
4-Diisopropylamino-2-(3-hydroxymethyl-phenyl)-2-phenyl-butyric acid ethyl ester;
4-Diisopropylamino-2-(3-hydroxymethyl-phenyl)-2-phenyl-butyric acid isopropyl ester; 2-(3-Acetoxymethyl-phenyl)-4-diisopropylamino-2-phenyl-butyric acid methyl ester.
5-Diisopropylamino-3,3-diphenyl-pentanoic acid;
5-Diisopropylamino-3,3-diphenyl-pentanoic acid methyl ester;
5-Diisopropylamino-3,3-diphenyl-pentanoic acid ethyl ester;
5-Diethylamino-3,3-diphenyl-pentanoic acid ethyl ester;
5-Diethylamino-2-methyl-3,3-diphenyl-pentanoic acid ethyl ester;
5-Diethylamino-2,2-dimethyl-3,3-diphenyl-pentanoic acid ethyl ester;
3-Cyclopentyl-5-diethylamino-2,2-dimethyl-3-phenyl-pentanoic acid ethyl ester;
3-Cyclohexyl-5-diethylamino-2,2-dimethyl-3-phenyl-pentanoic acid ethyl ester;
5-Diethylamino-3-hydroxy-2,2-dimethyl-3-phenyl-pentanoic acid ethyl ester;
1-(3-Diethylamino-1-hydroxy-1-phenyl-propyl)-cyclopentanecarboxylic acid methylester;
1-(3-Diethylamino-1-hydroxy-1-phenyl-propyl)-cyclohexanecarboxylic acid methyl ester;
1-(3-Diethylamino-1-hydroxy-1-phenyl-propyl)-cyclopropanecarboxylic acid methyl ester;
1-(5-Diethylamino-1-hydroxy-1-phenyl-pent-3-ynyl)-cyclohexanecarboxylic acid methyl ester;
1-(1-Hydroxy-1-phenyl-5-pyrrolidin-1-yl-pent-3-ynyl)-cyclohexanecarboxylic acid methyl ester; and
1-(1-Hydroxy-1-phenyl-5-pyrrolidin-1-yl-pent-3-ynyl)-cyclopentanecarboxylic acid methyl ester.

9. The compound, according to claim 2, wherein m, n, and p=0; X is a bond; and R₂ is hydroxyl.

10. The compound, according to claim 9, wherein said compound is selected from the group consisting of (3-Acetoxymethyl-phenyl)-hydroxy-phenyl-acetic acid 4-diethylamino-but-2-ynyl ester;
3-[(4-Diethylamino-but-2-ynyloxycarbonyl)-hydroxy-phenyl-methyl]-benzoic acid;
3-[(4-Diethylamino-but-2-ynyloxycarbonyl)-hydroxy-phenyl-methyl]-benzoic acid methyl ester;
3-[Hydroxy-(8-methyl-8-aza-bicyclo[3.2.1]oct-3-yloxycarbonyl)-phenyl-methyl]-benzoic acid methyl ester;
3-[2-Hydroxy-2-(3-methoxycarbonyl-phenyl)-2-phenyl-acetoxy]-8,8-dimethyl-8-azonia-bicyclo[3.2.1]octane; and
3-[(1-Aza-bicyclo[2.2.2]oct-3-yloxycarbonyl)-hydroxy-phenyl-methyl]-benzoic acid methyl ester.

11. The compound, according to claim 2, wherein m, n, and p=0; and R₂ and R₃ together form a cycloalkyl group.

12. The compound, according to claim 11, wherein said compound is selected from the group consisting of 3-[1-(1-Aza-bicyclo[2.2.2]oct-3-yloxycarbonyl)-cyclopentyl]-benzoic acid methyl ester;
3-[1-(1-Aza-bicyclo[2.2.2]oct-3-yloxycarbonyl)-cyclopentyl]-4-hydroxy-benzoic acid methyl ester;
3-[1-(1-Aza-bicyclo[2.2.2]oct-3-yloxycarbonyl)-cyclohexyl]-4-hydroxy-benzoic acid methyl ester;
3-[1-(4-Diethylamino-but-2-ynyloxycarbonyl)-cyclohexyl]-4-hydroxy-benzoic acid methyl ester;
3-[1-(4-Diethylamino-but-2-ynyloxycarbonyl)-cyclopentyl]-4-hydroxy-benzoic acid methyl ester;
4-Hydroxy-3-[1-(8-methyl-8-aza-bicyclo[3.2.1]oct-3-yloxycarbonyl)-cyclopentyl]-benzoic acid methyl ester; and
3-[1-(2-Hydroxy-5-methoxycarbonyl-phenyl)-cyclopentanecarbonyloxy]-8,8-dimethyl-8-azonia-bicyclo[3.2.1]octane.

13. The compound, according to claim 2, wherein X is not a bond.

14. The compound, according to claim 13, wherein said compound is selected from the group consisting of Cyclohexyl-(4-diethylamino-but-2-ynyloxycarbonyloxy)-phenyl-acetic acid;
Cyclohexyl-(4-diethylamino-but-2-ynyloxycarbonyloxy)-phenyl-acetic acid methyl ester;
Cyclohexyl-(4-diethylamino-but-2-ynyloxycarbonylamino)-phenyl-acetic acid methyl ester;
Cyclohexyl-(4-diethylamino-but-2-ynyloxycarbonylsulfanyl)-phenyl-acetic acid methyl ester;
(4-Diethylamino-but-2-ynyloxycarbonyloxy)-phenyl-acetic acid methyl ester;
(4-Diethylamino-but-2-ynyloxycarbonyloxy)-phenyl-acetic acid ethyl ester;
Phenyl-(4-pyrrolidin-1-yl-but-2-ynyloxycarbonyloxy)-acetic acid ethyl ester;
(8-Methyl-8-aza-bicyclo[3.2.1]oct-3-yloxycarbonyloxy)-phenyl-acetic acid ethyl ester;
3-(Ethoxycarbonyl-phenyl-methoxycarbonyloxy)-8,8-dimethyl-8-azonia-bicyclo[3.2.1]octane;
[(1-Aza-bicyclo[2.2.2]oct-3-yloxycarbonyloxy)]-phenyl-acetic acid ethyl ester;
3-(Ethoxycarbonyl-phenyl-methoxycarbonyloxy)-1-methyl-1-azonia-bicyclo[2.2.2]octane;
3-[2-Hydroxy-1-(3-methoxycarbonyl-phenyl)-ethoxycarbonyloxy]-1-methyl-1-azonia-bicyclo[2.2.2]octane;
3-[1-(1-Aza-bicyclo[2.2.2]oct-3-yloxycarbonyloxy)-2-hydroxy-ethyl]-benzoic acid methyl ester;
3-[2-Hydroxy-1-(8-methyl-8-aza-bicyclo[3.2.1]oct-3-yloxycarbonyloxy)-ethyl]-benzoic acid methyl ester; and
3-[2-Hydroxy-1-(3-methoxycarbonyl-phenyl)-ethoxycarbonyloxy]-8,8-dimethyl-8-azonia-bicyclo[3.2.1]octane.

15. A pharmaceutical composition comprising an anticholinergic compound having at least one of the following characteristics:
a. the compound is metabolized both by CYP450 and by a non-oxidative metabolic enzyme or system of enzymes;
b. the compound has a short (up to four (4) hours) non-oxidative metabolic half-life;
c. the compound contains a hydrolysable bond that can be cleaved non-oxidatively by hydrolytic enzymes;
d. the primary metabolites of the compound result from the non-oxidative metabolism of the compound;
e. the primary metabolites are soluble in water at physiological pH;
f. the primary metabolites have negligible inhibitory activity at the IK_{R} (HERG) channel at normal therapeutic concentration of the parent drug in plasma;
g. the compound, as well as the metabolites thereof, does not cause metabolic DDI when co-administered with other drugs; and
h. the compound, as well as metabolites thereof, does not elevate LFT values when administered alone;
wherein said composition comprises a pharmaceutical carrier.

16. The composition, according to claim 1, having the following formula: 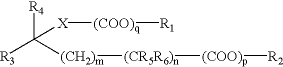
wherein:
R₁ is alkyl, alkenyl, alkynyl, cycloalkyl, cycloalkenyl, cycloalkynyl, or a combination thereof, containing at least one tertiary or quaternary nitrogen atom;
R₂ is H, OH, or C₁₋₄ alkyl;
R₃ and R₄ are, independently, H, hydroxyl, alkyl, cycloalkyl, aryl, or heteroaryl, optionally substituted with lower alkyl, hydroxyl, hydroxymethyl, COOH, or COO-lower alkyl;
R₅ and R₆ are independently H, C₁₋₄ alkyl, or R₅ and R₆ together form a cycloalkyl ring optionally containing a nitrogen or an oxygen atom, and optionally substituted with hydroxyl, hydroxymethyl, or lower alkyl;
m is an integer from 0 to 14;
n, p, and q are, independently, 0 or 1;
when m, n, and p=0, then R₂ and R₃ together can form a cycloalkyl ring optionally substituted with lower alkyl, COOH, or COO-lower alkyl; and
X is O, NH, S, CH₂, or X is a bond.

17. The composition, according to claim 16, wherein m, n, p, and q=0, X is a bond and R₂ is hydroxyl or hydrogen.

18. The composition, according to claim 17, wherein said compound is selected from the group consisting of 3-[3-Diisopropylamino-1-(2-hydroxy-phenyl)-propyl]-benzoic acid methyl ester;
3-[1-(2-Hydroxy-phenyl)-3-pyrrolidin-1-yl-propyl]-benzoic acid methyl ester;
3-(1-Cyclohexyl-3-diisopropylaminopropyl)-4-hydroxy-benzoic acid methyl ester;
Carboxymethyl-[3-cyclohexyl-3-(2-hydroxy-5-methyl-phenyl)-propyl]-diisopropyl-ammonium;
Methoxycarbonylmethyl-[3-cyclohexyl-3-(2-hydroxy-5-methyl-phenyl)-propyl]-diisopropyl-ammonium;
Methoxycarbonylmethyl-[3-(2-hydroxy-5-methyl-phenyl)-3-phenyl-propyl]-diisopropyl-ammonium;
Methoxycarbonylmethyl-[3-(5-acetoxymethyl-2-hydroxy-phenyl)-3-phenyl-propyl]-diisopropyl-ammonium.
3-(3-Diisopropylamino-1-hydroxy-1-phenyl-propyl)-benzoic acid;
3-(3-Diisopropylamino-1-hydroxy-1-phenyl-propyl)-benzoic acid methyl ester;
3-Diisopropylamino-1-(3-hydroxymethyl-phenyl)-1-phenyl-propan-1-ol;
3-Diisopropylamino-1-phenyl-1-m-tolyl-propan-1-ol;
Carboxymethyl-(3-hydroxy-3-phenyl-3-m-tolyl-propyl)-diisopropyl-ammonium; and
(3-Hydroxy-3-phenyl-3-m-tolyl-propyl)-diisopropyl-methoxycarbonylmethyl-ammonium.

19. The composition, according to claim 16, wherein m, n, p, and q=0; X is CH₂; and R₂ is hydroxyl.

20. The composition, according to claim 19, wherein said compound is selected from the groups consisting of 3-(3-Diisopropylamino-1-hydroxymethyl-1-phenyl-propyl)-benzoic acid;
3-(3-Diisopropylamino-1-hydroxymethyl-1-phenyl-propyl)-benzoic acid methyl ester;
4-Diisopropylamino-2-(3-hydroxymethyl-phenyl)-2-phenyl-butan-1-ol;
4-Diisopropylamino-2-phenyl-2-m-tolyl-butan-1-ol;
Carboxymethyl-(3-hydroxymethyl-3-phenyl-3-m-tolyl-propyl)-diisopropyl-ammonium; and
Methoxycarbonylmethyl-(3-hydroxymethyl-3-phenyl-3-m-tolyl-propyl)-diisopropyl-ammonium.

21. The composition, according to claim 16, wherein m, n, and q=0; p=1; X is a bond; and R₂ is hydrogen or lower alkyl.

22. The composition, according to claim 21, wherein said compound is selected from the group consisting of 4-Diisopropylamino-2,2-diphenyl-butyric acid;
4-Diisopropylamino-2,2-diphenyl-butyric acid methyl ester'
4-Diisopropylamino-2,2-diphenyl-butyric acid ethyl ester;
4-Diisopropylamino-2-phenyl-2-m-tolyl-butyric acid ethyl ester;
4-Diisopropylamino-2-(3-hydroxymethyl-phenyl)-2-phenyl-butyric acid ethyl ester;
4-Diisopropylamino-2-(3-hydroxymethyl-phenyl)-2-phenyl-butyric acid isopropyl ester; 2-(3-Acetoxymethyl-phenyl)-4-diisopropylamino-2-phenyl-butyric acid methyl ester.
5-Diisopropylamino-3,3-diphenyl-pentanoic acid;
5-Diisopropylamino-3,3-diphenyl-pentanoic acid methyl ester;
5-Diisopropylamino-3,3-diphenyl-pentanoic acid ethyl ester;
5-Diethylamino-3,3-diphenyl-pentanoic acid ethyl ester;
5-Diethylamino-2-methyl-3,3-diphenyl-pentanoic acid ethyl ester;
5-Diethylamino-2,2-dimethyl-3,3-diphenyl-pentanoic acid ethyl ester;
3-Cyclopentyl-5-diethylamino-2,2-dimethyl-3-phenyl-pentanoic acid ethyl ester;
3-Cyclohexyl-5-diethylamino-2,2-dimethyl-3-phenyl-pentanoic acid ethyl ester;
5-Diethylamino-3-hydroxy-2,2-dimethyl-3-phenyl-pentanoic acid ethyl ester;
1-(3-Diethylamino-1-hydroxy-1-phenyl-propyl)-cyclopentanecarboxylic acid methylester;
1-(3-Diethylamino-1-hydroxy-1-phenyl-propyl)-cyclohexanecarboxylic acid methyl ester;
1-(3-Diethylamino-1-hydroxy-1-phenyl-propyl)-cyclopropanecarboxylic acid methyl ester;
1-(5-Diethylamino-1-hydroxy-1-phenyl-pent-3-ynyl)-cyclohexanecarboxylic acid methyl ester;
1-(1-Hydroxy-1-phenyl-5-pyrrolidin-1-yl-pent-3-ynyl)-cyclohexanecarboxylic acid methyl ester; and
1-(1-Hydroxy-1-phenyl-5-pyrrolidin-1-yl-pent-3-ynyl)-cyclopentanecarboxylic acid methyl ester.

23. The composition, according to claim 16, wherein m, n, and p=0; X is a bond; and R₂ is hydroxyl.

24. The composition, according to claim 23, wherein said compound is selected from the group consisting of (3-Acetoxymethyl-phenyl)-hydroxy-phenyl-acetic acid 4-diethylamino-but-2-ynyl ester;
3-[(4-Diethylamino-but-2-ynyloxycarbonyl)-hydroxy-phenyl-methyl]-benzoic acid;
3-[(4-Diethylamino-but-2-ynyloxycarbonyl)-hydroxy-phenyl-methyl]-benzoic acid methyl ester;
3-[Hydroxy-(8-methyl-8-aza-bicyclo[3.2.1]oct-3-yloxycarbonyl)-phenyl-methyl]-benzoic acid methyl ester;
3-[2-Hydroxy-2-(3-methoxycarbonyl-phenyl)-2-phenyl-acetoxy]-8,8-dimethyl-8-azonia-bicyclo[3.2.1]octane; and
3-[(1-Aza-bicyclo[2.2.2]oct-3-yloxycarbonyl)-hydroxy-phenyl-methyl]-benzoic acid methyl ester.

25. The composition, according to claim 16, wherein m, n, and p=0; and R₂ and R₃ together form a cycloalkyl group.

26. The composition, according to claim 25, wherein said compound is selected from the group consisting of 3-[1-(1-Aza-bicyclo[2.2.2]oct-3-yloxycarbonyl)-cyclopentyl]-benzoic acid methyl ester;
3-[1-(1-Aza-bicyclo[2.2.2]oct-3-yloxycarbonyl)-cyclopentyl]-4-hydroxy-benzoic acid methyl ester;
3-[1-(1-Aza-bicyclo[2.2.2]oct-3-yloxycarbonyl)-cyclohexyl]-4-hydroxy-benzoic acid methyl ester;
3-[1-(4-Diethylamino-but-2-ynyloxycarbonyl)-cyclohexyl]-4-hydroxy-benzoic acid methyl ester;
3-[1-(4-Diethylamino-but-2-ynyloxycarbonyl)-cyclopentyl]-4-hydroxy-benzoic acid methyl ester;
4-Hydroxy-3-[1-(8-methyl-8-aza-bicyclo[3.2.1]oct-3-yloxycarbonyl)-cyclopentyl]-benzoic acid methyl ester; and
3-[1-(2-Hydroxy-5-methoxycarbonyl-phenyl)-cyclopentanecarbonyloxy]-8,8-dimethyl-8-azonia-bicyclo[3.2.1]octane.

27. The compound, according to claim 16, wherein X is not a bond.

28. The composition, according to claim 27, wherein said compound is selected from the group consisting of Cyclohexyl-(4-diethylamino-but-2-ynyloxycarbonyloxy)-phenyl-acetic acid;
Cyclohexyl-(4-diethylamino-but-2-ynyloxycarbonyloxy)-phenyl-acetic acid methyl ester;
Cyclohexyl-(4-diethylamino-but-2-ynyloxycarbonylamino)-phenyl-acetic acid methyl ester;
Cyclohexyl-(4-diethylamino-but-2-ynyloxycarbonylsulfanyl)-phenyl-acetic acid methyl ester;
(4-Diethylamino-but-2-ynyloxycarbonyloxy)-phenyl-acetic acid methyl ester;
(4-Diethylamino-but-2-ynyloxycarbonyloxy)-phenyl-acetic acid ethyl ester;
Phenyl-(4-pyrrolidin-1-yl-but-2-ynyloxycarbonyloxy)-acetic acid ethyl ester;
(8-Methyl-8-aza-bicyclo[3.2.1]oct-3-yloxycarbonyloxy)-phenyl-acetic acid ethyl ester;
3-(Ethoxycarbonyl-phenyl-methoxycarbonyloxy)-8,8-dimethyl-8-azonia-bicyclo[3.2.1]octane;
[(1-Aza-bicyclo[2.2.2]oct-3-yloxycarbonyloxy)]-phenyl-acetic acid ethyl ester;
3-(Ethoxycarbonyl-phenyl-methoxycarbonyloxy)-1-methyl-1-azonia-bicyclo[2.2.2]octane;
3-[2-Hydroxy-1-(3-methoxycarbonyl-phenyl)-ethoxycarbonyloxy]-1-methyl-1-azonia-bicyclo[2.2.2]octane;
3-[1-(1-Aza-bicyclo[2.2.2]oct-3-yloxycarbonyloxy)-2-hydroxy-ethyl]-benzoic acid methyl ester;
3-[2-Hydroxy-1-(8-methyl-8-aza-bicyclo[3.2.1]oct-3-yloxycarbonyloxy)-ethyl]-benzoic acid methyl ester; and
3-[2-Hydroxy-1-(3-methoxycarbonyl-phenyl)-ethoxycarbonyloxy]-8,8-dimethyl-8-azonia-bicyclo[3.2.1]octane.

29. A method for providing anticholinergic activity to a patient in need of such activity wherein said method comprises administering to said patient an anticholinergic compound having at least one of the following characteristics:
a. the compound is metabolized both by CYP450 and by a non-oxidative metabolic enzyme or system of enzymes;
b. the compound has a short (up to four (4) hours) non-oxidative metabolic half-life;
c. the compound contains a hydrolysable bond that can be cleaved non-oxidatively by hydrolytic enzymes;
d. the primary metabolites of the compound result from the non-oxidative metabolism of the compound;
e. the primary metabolites are soluble in water at physiological pH;
f. the primary metabolites have negligible inhibitory activity at the IK_{R} (HERG) channel at normal therapeutic concentration of the parent drug in plasma;
g. the compound, as well as the metabolites thereof, does not cause metabolic DDI when co-administered with other drugs; and
h. the compound, as well as metabolites thereof, does not elevate LFT values when administered alone.

30. The method, according to claim 29, wherein said compound has the following formula: 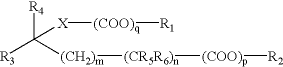
wherein:
R₁ is alkyl, alkenyl, alkynyl, cycloalkyl, cycloalkenyl, cycloalkynyl, or a combination thereof, containing at least one tertiary or quaternary nitrogen atom;
R₂ is H, OH, or C₁₋₄ alkyl;
R₃ and R₄ are, independently, H, hydroxyl, alkyl, cycloalkyl, aryl, or heteroaryl, optionally substituted with lower alkyl, hydroxyl, hydroxymethyl, COOH, or COO-lower alkyl;
R₅ and R₆ are independently H, C₁₋₄ alkyl, or R₅ and R₆ together form a cycloalkyl ring optionally containing a nitrogen or an oxygen atom, and optionally substituted with hydroxyl, hydroxymethyl, or lower alkyl;
m is an integer from 0 to 14;
n, p, and q are, independently, 0 or 1;
when m, n, and p=0, then R₂ and R₃ together can form a cycloalkyl ring optionally substituted with lower alkyl, COOH, or COO-lower alkyl; and
X is O, NH, S, CH₂, or X is a bond.

31. The method, according to claim 30, wherein m, n, p, and q=0, X is a bond and R₂ is hydroxyl or hydrogen.

32. The method, according to claim 31, wherein said compound is selected from the group consisting of 3-[3-Diisopropylamino-1-(2-hydroxy-phenyl)-propyl]-benzoic acid methyl ester;
3-[1-(2-Hydroxy-phenyl)-3-pyrrolidin-1-yl-propyl]-benzoic acid methyl ester;
3-(1-Cyclohexyl-3-diisopropylaminopropyl)-4-hydroxy-benzoic acid methyl ester;
Carboxymethyl-[3-cyclohexyl-3-(2-hydroxy-5-methyl-phenyl)-propyl]-diisopropyl-ammonium;
Methoxycarbonylmethyl-[3-cyclohexyl-3-(2-hydroxy-5-methyl-phenyl)-propyl]-diisopropyl-ammonium;
Methoxycarbonylmethyl-[3-(2-hydroxy-5-methyl-phenyl)-3-phenyl-propyl]-diisopropyl-ammonium;
Methoxycarbonylmethyl-[3-(5-acetoxymethyl-2-hydroxy-phenyl)-3-phenyl-propyl]-diisopropyl-ammonium.
3-(3-Diisopropylamino-1-hydroxy-1-phenyl-propyl)-benzoic acid;
3-(3-Diisopropylamino-1-hydroxy-1-phenyl-propyl)-benzoic acid methyl ester;
3-Diisopropylamino-1-(3-hydroxymethyl-phenyl)-1-phenyl-propan-1-ol;
3-Diisopropylamino-1-phenyl-1-m-tolyl-propan-1-ol;
Carboxymethyl-(3-hydroxy-3-phenyl-3-m-tolyl-propyl)-diisopropyl-ammonium; and
(3-Hydroxy-3-phenyl-3-m-tolyl-propyl)-diisopropyl-methoxycarbonylmethyl-ammonium.

33. The method, according to claim 30, wherein m, n, p, and q=0; X is CH₂; and R₂ is hydroxyl.

34. The method, according to claim 33, wherein said compound is selected from the groups consisting of 3-(3-Diisopropylamino-1-hydroxymethyl-1-phenyl-propyl)-benzoic acid;
3-(3-Diisopropylamino-1-hydroxymethyl-1-phenyl-propyl)-benzoic acid methyl ester;
4-Diisopropylamino-2-(3-hydroxymethyl-phenyl)-2-phenyl-butan-1-ol;
4-Diisopropylamino-2-phenyl-2-m-tolyl-butan-1-ol;
Carboxymethyl-(3-hydroxymethyl-3-phenyl-3-m-tolyl-propyl)-diisopropyl-ammonium; and
Methoxycarbonylmethyl-(3-hydroxymethyl-3-phenyl-3-m-tolyl-propyl)-diisopropyl-ammonium.

35. The method, according to claim 30, wherein m, n, and q=0; p=1; X is a bond; and R₂ is hydrogen or lower alkyl.

36. The method, according to claim 35, wherein said compound is selected from the group consisting of 4-Diisopropylamino-2,2-diphenyl-butyric acid;
4-Diisopropylamino-2,2-diphenyl-butyric acid methyl ester'
4-Diisopropylamino-2,2-diphenyl-butyric acid ethyl ester;
4-Diisopropylamino-2-phenyl-2-m-tolyl-butyric acid ethyl ester;
4-Diisopropylamino-2-(3-hydroxymethyl-phenyl)-2-phenyl-butyric acid ethyl ester;
4-Diisopropylamino-2-(3-hydroxymethyl-phenyl)-2-phenyl-butyric acid isopropyl ester; 2-(3-Acetoxymethyl-phenyl)-4-diisopropylamino-2-phenyl-butyric acid methyl ester.
5-Diisopropylamino-3,3-diphenyl-pentanoic acid;
5-Diisopropylamino-3,3-diphenyl-pentanoic acid methyl ester;
5-Diisopropylamino-3,3-diphenyl-pentanoic acid ethyl ester;
5-Diethylamino-3,3-diphenyl-pentanoic acid ethyl ester;
5-Diethylamino-2-methyl-3,3-diphenyl-pentanoic acid ethyl ester;
5-Diethylamino-2,2-dimethyl-3,3-diphenyl-pentanoic acid ethyl ester;
3-Cyclopentyl-5-diethylamino-2,2-dimethyl-3-phenyl-pentanoic acid ethyl ester;
3-Cyclohexyl-5-diethylamino-2,2-dimethyl-3-phenyl-pentanoic acid ethyl ester;
5-Diethylamino-3-hydroxy-2,2-dimethyl-3-phenyl-pentanoic acid ethyl ester;
1-(3-Diethylamino-1-hydroxy-1-phenyl-propyl)-cyclopentanecarboxylic acid methylester;
1-(3-Diethylamino-1-hydroxy-1-phenyl-propyl)-cyclohexanecarboxylic acid methyl ester;
1-(3-Diethylamino-1-hydroxy-1-phenyl-propyl)-cyclopropanecarboxylic acid methyl ester;
1-(5-Diethylamino-1-hydroxy-1-phenyl-pent-3-ynyl)-cyclohexanecarboxylic acid methyl ester;
1-(1-Hydroxy-1-phenyl-5-pyrrolidin-1-yl-pent-3-ynyl)-cyclohexanecarboxylic acid methyl ester; and
1-(1-Hydroxy-1-phenyl-5-pyrrolidin-1-yl-pent-3-ynyl)-cyclopentanecarboxylic acid methyl ester.

37. The method, according to claim 30, wherein m, n, and p=0; X is a bond; and R₂ is hydroxyl.

38. The method, according to claim 37, wherein said compound is selected from the group consisting of (3-Acetoxymethyl-phenyl)-hydroxy-phenyl-acetic acid 4-diethylamino-but-2-ynyl ester;
3-[(4-Diethylamino-but-2-ynyloxycarbonyl)-hydroxy-phenyl-methyl]-benzoic acid;
3-[(4-Diethylamino-but-2-ynyloxycarbonyl)-hydroxy-phenyl-methyl]-benzoic acid methyl ester;
3-[Hydroxy-(8-methyl-8-aza-bicyclo[3.2.1]oct-3-yloxycarbonyl)-phenyl-methyl]-benzoic acid methyl ester;
3-[2-Hydroxy-2-(3-methoxycarbonyl-phenyl)-2-phenyl-acetoxy]-8,8-dimethyl-8-azonia-bicyclo[3.2.1]octane; and
3-[(1-Aza-bicyclo[2.2.2]oct-3-yloxycarbonyl)-hydroxy-phenyl-methyl]-benzoic acid methyl ester.

39. The method, according to claim 30, wherein m, n, and p=0; and R₂ and R₃ together form a cycloalkyl group.

40. The method, according to claim 39, wherein said compound is selected from the group consisting of 3-[1-(1-Aza-bicyclo[2.2.2]oct-3-yloxycarbonyl)-cyclopentyl]-benzoic acid methyl ester;
3-[1-(1-Aza-bicyclo[2.2.2]oct-3-yloxycarbonyl)-cyclopentyl]-4-hydroxy-benzoic acid methyl ester;
3-[1-(1-Aza-bicyclo[2.2.2]oct-3-yloxycarbonyl)-cyclohexyl]-4-hydroxy-benzoic acid methyl ester;
3- [1-(4-Diethylamino-but-2-ynyloxycarbonyl)-cyclohexyl]-4-hydroxy-benzoic acid methyl ester;
3-[1-(4-Diethylamino-but-2-ynyloxycarbonyl)-cyclopentyl]-4-hydroxy-benzoic acid methyl ester;
4-Hydroxy-3-[1-(8-methyl-8-aza-bicyclo[3.2.1]oct-3-yloxycarbonyl)-cyclopentyl]-benzoic acid methyl ester; and
3-[1-(2-Hydroxy-5-methoxycarbonyl-phenyl)-cyclopentanecarbonyloxy]-8,8-dimethyl-8-azonia-bicyclo[3.2.1]octane.

41. The compound, according to claim 30, wherein X is not a bond.

42. The compound, according to claim 41, wherein said compound is selected from the group consisting of Cyclohexyl-(4-diethylamino-but-2-ynyloxycarbonyloxy)-phenyl-acetic acid;
Cyclohexyl-(4-diethylamino-but-2-ynyloxycarbonyloxy)-phenyl-acetic acid methyl ester;
Cyclohexyl-(4-diethylamino-but-2-ynyloxycarbonylamino)-phenyl-acetic acid methyl ester;
Cyclohexyl-(4-diethylamino-but-2-ynyloxycarbonylsulfanyl)-phenyl-acetic acid methyl ester;
(4-Diethylamino-but-2-ynyloxycarbonyloxy)-phenyl-acetic acid methyl ester;
(4-Diethylamino-but-2-ynyloxycarbonyloxy)-phenyl-acetic acid ethyl ester;
Phenyl-(4-pyrrolidin-1-yl-but-2-ynyloxycarbonyloxy)-acetic acid ethyl ester;
(8-Methyl-8-aza-bicyclo[3.2.1]oct-3-yloxycarbonyloxy)-phenyl-acetic acid ethyl ester;
3-(Ethoxycarbonyl-phenyl-methoxycarbonyloxy)-8,8-dimethyl-8-azonia-bicyclo[3.2.1]octane;
[(1-Aza-bicyclo[2.2.2]oct-3-yloxycarbonyloxy)]-phenyl-acetic acid ethyl ester;
3-(Ethoxycarbonyl-phenyl-methoxycarbonyloxy)-1-methyl-1-azonia-bicyclo[2.2.2]octane;
3-[2-Hydroxy-1-(3-methoxycarbonyl-phenyl)-ethoxycarbonyloxy]-1-methyl-1-azonia-bicyclo[2.2.2]octane;
3-[1-(1-Aza-bicyclo[2.2.2]oct-3-yloxycarbonyloxy)-2-hydroxy-ethyl]-benzoic acid methyl ester;
3-[2-Hydroxy-1-(8-methyl-8-aza-bicyclo[3.2.1]oct-3-yloxycarbonyloxy)-ethyl]-benzoic acid methyl ester; and
3-[2-Hydroxy-1-(3-methoxycarbonyl-phenyl)-ethoxycarbonyloxy]-8,8-dimethyl-8-azonia-bicyclo[3.2.1]octane.

43. The method, according to claim 29, used to treat incontinence.

44. The method, according to claim 29, used to create bronchodilation.

45. The method, according to claim 44, used to treat asthma or obstructive airway disease.

46. The method, according to claim 29, wherein the compound is used as a mydriatic agent.

47. The method, according to claim 29, wherein the compound is used as an anti-perspirant.

48. The method, according to claim 29, wherein the patient is a human.
